(19) United States
(12) Patent Application Publication
(10) Pub. No.: US 2008/0004495 A1
(43) Pub. Date: Jan. 3, 2008

(54) ENDOSCOPE

(75) Inventors: John David Allen, Essendon (AU); Peter Maxwell Delaney, Carnegie (AU)

Correspondence Address: KNOBBE MARTENS OLSON & BEAR LLP, 2040 MAIN STREET, FOURTEENTH FLOOR, IRVINE, CA, 92614, US

(73) Assignee: Optiscan Pty Ltd., Notting Hill (AU)

(21) Appl. No.: 11/753,506

(22) Filed: May 24, 2007

Related U.S. Application Data
(63) Continuation of application No. PCT/AU05/01782, filed on Nov. 24, 2005.

(30) Foreign Application Priority Data
Nov. 25, 2004 (AU) 2004906759

(51) Int. Cl. A61B 1/06

(52) U.S. Cl. 600/160

(57) ABSTRACT

An endoscope comprising a first light source for illuminating biological tissue with light, a first detector for detecting macroscopic images and fluorescent images from the tissue by reflected light and fluorescent light induced in the tissue, a second light source for illuminating the tissue with light, a confocal microscopic waveguide for supplying light from the second light source to the tissue and for supplying microscopic fluorescent images of the tissue, and a second detector for detecting the microscopic fluorescent images from the confocal microscopic waveguide.

### CROSS-REFERENCE TO RELATED APPLICATIONS

This application is a continuation of PCT Patent Application No. PCT/AU2005/001782 filed on Nov. 24, 2005 which claims priority of Australian Patent Application No. 2004906759 filed on Nov. 25, 2004, the disclosures of which are incorporated herein in their entirety by reference.

### BACKGROUND OF THE INVENTION

1. Field of the Invention

The present invention relates to an endoscope.

2. Description of the Related Art

Endoscopes are widely used today to image the internal lining of the gastro-intestinal tract for various disease and pathological states. Traditionally, normal light images are displayed in color and are obtained using a conventional white light source, the light from which is transmitted via an optical fiber to emerge from the tip of the endoscope. Light reflected from the tissue is picked up by a detector such as a CCD chip situated at the tip of the endoscope and the light is transmitted to an image processor so the image can be processed and displayed on a monitor. In even more recent times, both color and fluorescent images have been possible. However, only macroscopic changes to a region of the tissue being examined can be detected by such systems, whether operated in normal color mode or in the fluorescent image mode.

### SUMMARY OF THE INVENTION

In one embodiment, the invention provides an endoscope comprising:

a first light source for illuminating biological tissue—such as of a patient—with light;

a first detector for detecting macroscopic images and fluorescent images from the tissue by reflected light and fluorescent light induced in the tissue;

a second light source for illuminating the tissue with light;

a confocal microscopic waveguide for supplying the light from the second light source to the tissue and for supplying microscopic fluorescent images of the tissue; and

a second detector for detecting the microscopic fluorescent images from the confocal microscopic waveguide.

According to one embodiment, an endoscopist can observe macroscopic observable areas or lesions within the overall tissue under white light illumination and can switch to macroscopic fluorescent mode to observe areas within the overall structure where changes to cell population and architecture produce fluorescent images at a concentration that is different from surrounding tissue. These discrete areas would be further investigated by the operator taking biopsies of the area to investigate further by subsequent histopathology. The taking of such biopsies has some risk to the patient and the operator is not sure until the results of the biopsy are obtained some several days later whether the correct area was selected for biopsy, or the small area of tissue that was collected was representative of the greater area. Once having observed the area of the tissue of interest, the operator can switch to the confocal microscopic waveguide for detecting microscopic fluorescent images of the area. This enables the microscopic cell morphology and cellular architecture to be assessed in near real time during the actual procedure and a decision can be made, if warranted, that more extensive mucosal resections of the affected area should be undertaken during the same procedure to avoid rescheduling of the patient for a further procedure. Thus, the three imaging modes, namely macroscopic images, fluorescent images and microscopic fluorescent images offer an increased sensitivity for the operator to macroscopically detect the presence of small abnormal lesions and then to microscopically observe these detected lesions to determine the nature of the cell morphology and cellular structure (e.g. is consistent with normal structure, or is displaying dysplasia or early stage neoplasia), thereby allowing the operator to have an increased specificity to classify a lesion and, in turn, the choice of appropriate actions (e.g. removal). The invention also therefore provides for greater accuracy of patient diagnosis.

In one aspect, a waveguide is provided for supplying the light from the first light source to the tissue.

In one embodiment, the first light source is a white light source and includes a filter for rapidly changing the effective illumination through the visible spectrum such that different tissue structures being illuminated by the changing wavelengths of the illumination light reflect varying amounts of light to the first detector.

In this embodiment the detector is a monochrome detector.

In another embodiment, the light source may be a continuous white light source and the first detector may be a color detector for detecting various amounts of the different wavelengths of reflected light.

In this embodiment the waveguide comprises a single fiber or a fiber bundle for conducting light from the first light source to the tissue and the first detector is at a free end of the endoscope.

In a further embodiment the waveguide comprises a first waveguide for conducting light to a free end of the endoscope and a second waveguide for receiving reflected light and conveying the reflected light to the first detector.

In this embodiment the first detector is located remote from the free end of the endoscope and a further filter may be provided at the free end of the endoscope for filtering the light received by the second waveguide or the further filter may be located between the detector and the second waveguide.

As in the previous embodiments, the first detector may be a monochrome detector or a color detector and may include the filter for filtering light from the first light source before the light is provided to the first waveguide.

The first and second waveguides may be single fibers or fiber bundles.

The confocal microscope waveguide may be a single fiber or fiber bundle.

In one embodiment the confocal microscope fiber or fiber bundle includes a fiber coupler for receiving the fluorescent light and directing the fluorescent light to a second detector.

In one aspect, a monitor is provided for providing a display which comprises an overlap of the macroscopic and fluorescent images to produce a single image.

The second light source is for producing light at a predetermined monochrome wavelength for passage through the confocal microscope waveguide.

In one embodiment, the second light source comprises a laser light source.

In one embodiment the first detector is connected to a processor and the detector includes a plurality of different color chips so that the intensity gains for the different color chips in the first detector can be adjusted to maximize the detection of returning fluorescent light and reduce the detection of any background reflected light.

The second detector is also connected to the processor so that the processor can process the images and display on a monitor the light image, the macroscopic fluorescent image and the microscopic fluorescent image.

The fluorescent light is induced in the tissue by administering an exogenous fluorescent contrast agent to the patient. The contrast agent may be sodium fluorescein (NaF) which is administered by intravenous injection to the patient at the time that the endoscope is inserted.

However, other contrast agents could be used if desired.

Another embodiment provides a method of inspecting a patient's tissue by use of an endoscope, comprising:

applying an exogenous contrast agent to the patient;

illuminating the patient's tissue with the endoscope;

detecting with the endoscope a light image of the tissue by the endoscope;

detecting with the endoscope a macroscopic fluorescent image; and

detecting with the endoscope a microscopic confocal fluorescent image of the tissue.

In one embodiment, the endoscope includes a filter and a color detector and filter parameters and gain of the color detector are controlled to be at the maximum excitation and emission peaks respectively for the contrast agent administered to the patient.

In one aspect, the method comprises inspecting the macroscopic fluorescent image to identify regions of interest and further inspecting microscopic confocal fluorescent images of those regions.

In one embodiment the contrast agent comprises sodium fluorescein. However, in other embodiments the agent may be acriflavine which is applied by the endoscope rather than by injection to the patient.

Another embodiment provides an endoscope comprising:

a selector mechanism configured to select between a plurality of modes of operation, the modes of operation comprising:

a white light color mode of operation in which a color image of a patient's tissue is obtained;

a macroscopic fluorescent mode in which a macroscopic fluorescent image of the tissue is obtained; and

a microscopic confocal fluorescent mode in which a microscopic fluorescent image of the tissue is obtained.

Another embodiment provides a method of obtaining images of a patient with a single endoscope comprising administering an exogenous contrast agent to the patient, and obtaining one or more of a white light color image, a macroscopic fluorescent image and a microscopic confocal fluorescent image of tissue of the patient.

It should be understood that, although the apparatus is termed an “endoscope”, this term is not intended to limit the apparatus to internal use, or to in vivo applications.

### BRIEF DESCRIPTION OF THE DRAWINGS

In order that the invention may be more clearly ascertained, certain embodiments of the invention will be described, by way of example, with reference to the accompanying drawing in which:

FIG. 1 is a view of a first embodiment;

FIG. 2 is a view of a second embodiment;

FIG. 3 is a view of a monitor and display system used in some embodiments;

FIGS. 4A, 4B and 4C are, respectively, examples (reproduced in greyscale) of a normal white light macroscopic image, a corresponding macroscopic fluorescence image and a confocal microscopic image, collected according to an embodiment; and

FIGS. 5A and 5B are, respectively, the original color versions of FIGS. 4A and 4B.

### DETAILED DESCRIPTION OF THE PREFERRED EMBODIMENTS

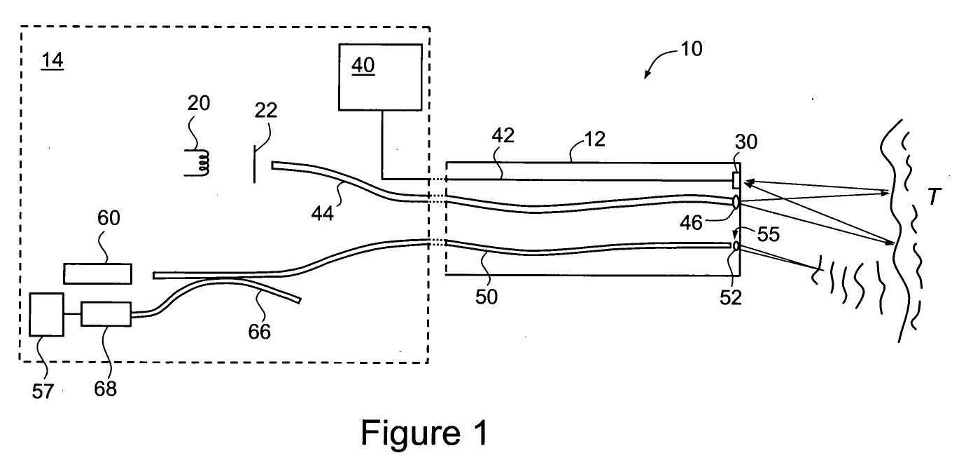

With reference to FIG. 1, an endoscope 10 is shown for inspecting tissue T of a patient. The endoscope 10 has an insertion section 12 and a light source and processing section 14.

The section 14 includes a bulb 20 for producing white light and may have a filter wheel 22 which is rapidly rotated in front of the bulb 20 so as to produce different wavelengths from red to blue to green to white light and then for repeating that cycle, so that different tissue structures being illuminated by the changing wavelengths of the light reflect varying amounts of light to a detector 30 at the free end of the insertion section 12. If the filter wheel 22 is used typically, the detector 30 is a monochrome detector, such as a monochrome CCD. In other embodiments, the filter wheel 22 can be omitted and the detector 30 could be a multi-chip color CCD for detecting the varying amounts of the different wavelengths reflected from the tissue T. The detector 30 is connected to a processor and monitor 40 via line 42 for processing the signals from the detector 30 and for displaying the images detected by the detector 30.

Light is conveyed from the bulb 20 by a waveguide 44 which may be a single optical fiber or a fiber bundle. An illumination lens 46 may be provided at the end of the fiber 44.

A biologically compatible fluorescent contrast agent, such as sodium fluorescein, is administered to the patient at the time of insertion of the endoscope 10. The agent can be applied systematically such as by injection, or topically from the end of the endoscope. In the case of sodium fluorescein, administration is usually by IV injection.

Typically the endoscopist will observe areas or lesions of the tissue T (such as the colon) under white light illumination, but can switch to macroscopic fluorescent mode to observe areas of the overall structure where changes to cell population and architecture means that the externally applied fluorescent contrast agent may partition within the various cell structures of the tissue to highlight the various different architectural or structural features of the tissue. These discrete areas with different accumulations or distributions of contrast agent may be invisible to the operator under white light color imaging, and their presence, once detected, alerts the operator to investigate further.

When operating the endoscope in the macroscopic fluorescent mode, the tissue is illuminated by light of a particular wavelength which may be obtained by rotating the filter wheel 22 to a particular position to produce light of that wavelength. For example, the light may be blue light. If the filter wheel 22 is omitted and a color CCD chip type detector 30 is used, then a blue filter may be interposed between the light source 20 and the fiber bundle 44 or, alternatively, a filter wheel which has a clear filter for allowing white light to pass and a blue filter may be used so that the filter wheel is rotated to bring the blue filter into position when operating in the macroscopic fluorescent mode.

If a monochrome CCD detector is used with the filter wheel 22, the filter wheel is stopped in a particular position (i.e. blue) and a second separate filter is switched at the end of the endoscope. This filter could be a green long pass filter so only the green fluorescence is detected and the blue reflected light is not detected.

The endoscope 10 further includes a confocal fiber 50 which has an objective lens set 52 at its end. The fiber 50 may be a single fiber of fiber bundle and is provided with light of a particular wavelength from a light source such as a laser 60. The fiber 50 has a single mode fiber coupler 66 for supplying returning light through the fiber 50 to a second detector 68. The detector 68 can then supply output to a processor for display of an image on a monitor. The processor and monitor may be the same as the processor and monitor 40 or may be a different processor and monitor.

Thus, according to an embodiment, once the operator has observed an area of the tissue by the macroscopic fluorescent mode where light is detected by the CCD 30 and observed an area of the tissue with a different accumulation of contrast agent, then instead of needing to take biopsies to investigate further, the operator can use the confocal fiber 50 and further investigate the cell morphology and cellular architecture at a microscopic level before deciding whether a biopsy is warranted. The operator can therefore assess the microscopic cell morphology and cellular architecture in near real time during the actual procedure and can decide, if warranted, that more extensive mucosal resections of the affected area should be undertaken during the same procedure to avoid rescheduling of the patient for a further procedure.

In one embodiment, the output from the detector 30 is controlled by the processor 40 by software so the monitor of the processor 40 displays a color image. The filter 22 can selectively be placed between the light source 20 and the fiber 44 so that the endoscope 10 operates in macroscopic fluorescent mode, where the detector 30 detects fluorescent light generated from the tissue. Typically, the blue filter previously mentioned is used to provide blue light. The agent which has accumulated or not accumulated in the tissue T and from which fluorescence is produced or not produced under the influence of the blue light can then be observed. If a color CCD detector is used, a software feedback loop is used so that the relative intensity gains for the different color chips in the detector 30 can be adjusted to maximize the detection of the returning fluorescent light and to reduce the detection of any background reflected light. Thus, the operator can then visualize the discrete areas in the tissue being examined that display differential fluorescence intensity and distribution. Those areas can then be further investigated by placing the confocal tip 55 of the confocal fiber 50 onto the appropriately selected areas on the surface of the tissue to obtain and review near real time confocal images which are software matched to the preceding macroscopic fluorescent images. Both sets of images can be digitally stored for later review if desired. Typically, the tip 55 includes lenses and a cover slip and, if a single fiber, can include a scanning device.

The laser 60 provides the blue light for illumination of the detected areas so that the fluorescent light is produced and collected at a microscopic level by the confocal fiber 50 to provide the microscopic fluorescent image of the selected area which can be displayed on a monitor 57.

In the case of sodium fluorescein, the blue filter allows passage of light of between 450 to 500 nanometer wavelength. The gain of the CCD detector 30 is set to maximize the green channel as sodium fluorescein emits a fluorescent signal with a peak of 513 nanometers. If other agents are used, then the filter is set to maximize their excitation peak and the gain for the chip adjusted to the maximum emission peak. Similarly, the wavelengths applied by the laser 60 is also selected to match the excitation peak of the agent being used.

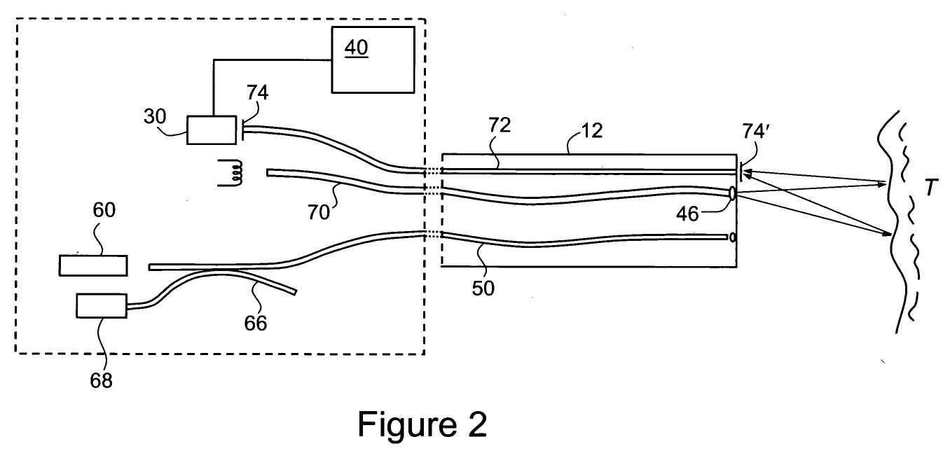

FIG. 2 shows a second embodiment in which like reference numerals indicate like parts to those previously described.

In FIG. 2, the light source 20 supplies light to a first waveguide 70 for illuminating the tissue T. Reflected light from the tissue T is received by a second waveguide 72 which conveys the light to the detector 30. The detector 30 may be the monochrome detector previously described or the color detector. If a monochrome detector is used, then the filter wheel 22 (not shown in FIG. 2) is disposed between the light source 20 and the first waveguide 70. When it is desired to obtain the macroscopic fluorescent image, the filter wheel is stopped at the predetermined position, such as the blue filter position, to provide blue light for inducing the fluorescence, which is then received by the waveguide 72 and conveyed to the detector 30. In this embodiment, a further filter 74 may be disposed between the waveguide 72 and the detector 30. The further filter 74 may be a long pass filter for providing wavelengths of the fluorescent wavelength which is induced by the blue light, such as a green filter. In some embodiments, the filter 74 may be located at the free end of the endoscope, as shown by reference 74′ in FIG. 2. The waveguides 70 and 72 may comprise single fibers or a fiber bundle.

When collecting the normal white light macroscopic image, the filter 74 is not in position and the detector 30 simply detects all of the wavelengths which are reflected from the tissue T, which may be the entire color band if a color detector 30 is used or sequentially various wavelengths as provided by the filter wheel 22 (not shown in FIG. 2). The image is then displayed as the color image on the monitor 40 or is built up from the various wavelength images if a monochrome detector 30 is used.

The confocal microscope waveguide 50 is configured in the same manner as in the previous embodiment and operates in the same way.

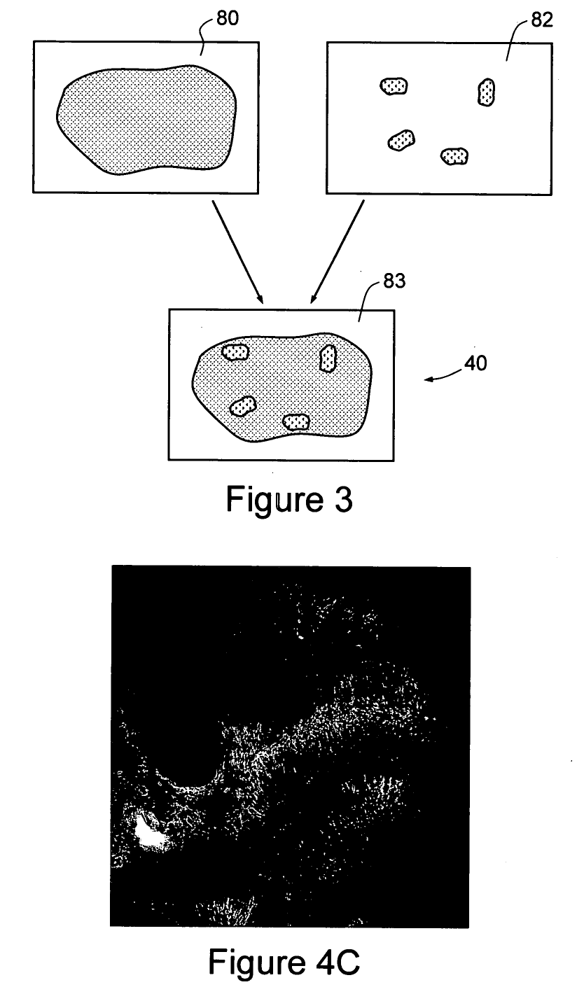

FIG. 3 is a view of the various images that are obtained according to an embodiment. In this embodiment, two monitors 40 and 57 (see FIG. 1) are used for the macroscopic images and the confocal microscopic image respectively. The normal white light image and the macroscopic fluorescent image may be overlaid to provide a single image so the various locations of the fluorescent image on the normal white light image can be seen. As is shown in FIG. 3, image 80 represents the macroscopic white light image and image 82 the macroscopic fluorescent image. The images are overlaid on monitor 40 by switching the various images to the monitor 40 by half the imaging rate of the monitor 40 so that both images are seen together as a single image 83. The manner in which the images 80 and 82 are overlaid to produce the single image can be performed in any desirable way.

Although, in one embodiment, a separate monitor is used for the microscopic fluorescent image, the monitor 40 may also be used for that image by a split screen technique so that part of the monitor 40 shows the microscopic fluorescent image and part of the monitor shows the overlaid macroscopic images. In still further embodiments, the split screen technique may be used to show the separate images 80 and 82, as well as the overlaid image 83 and then also the microscopic image. In still further embodiments, three separate monitors could also be used to show the three different images.

### EXAMPLES

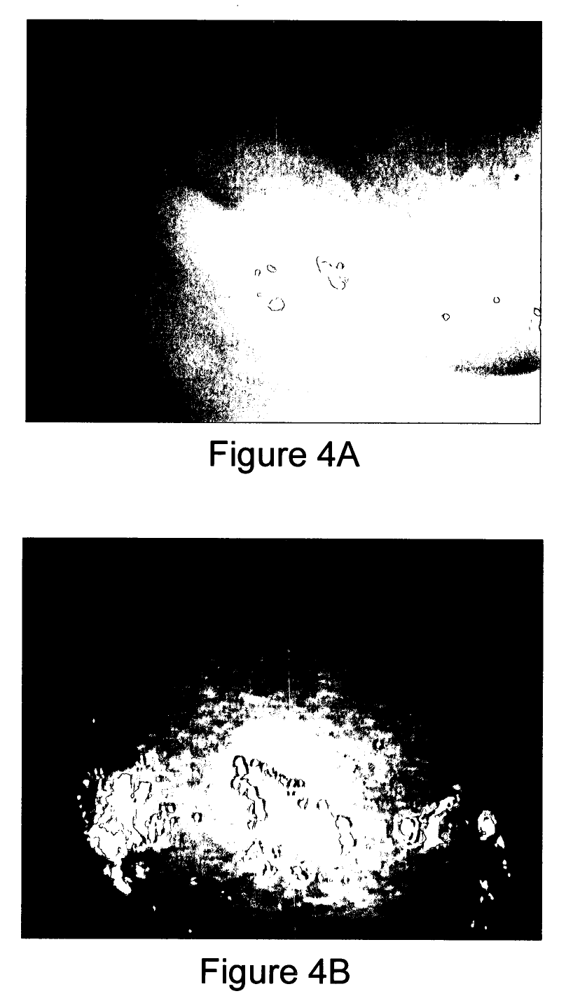

FIGS. 4A, 4B and 4C are, respectively, examples (reproduced in greyscale) of a normal white light macroscopic image (cf. image 80 of FIG. 3), a corresponding macroscopic fluorescence image (cf. image 82 of FIG. 3) and a confocal microscopic image, collected by means of endoscope 10 of FIG. 1. These images are of a portion of a human colon, and were collected following the administration by intravenous injection of a fluorescent contrast agent in the form of 5 mL of Pharmalab brand sodium fluorescein 10% solution.

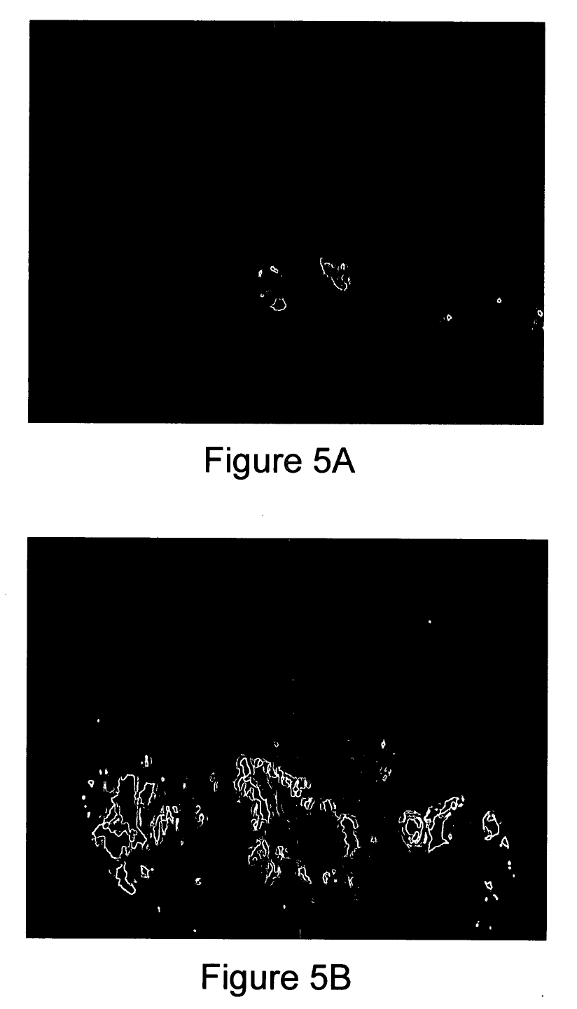

FIGS. 5A and 5B are, respectively, the original color versions of FIGS. 4A and 4B.

The images of FIGS. 4A and 4B are of the same portion of the colon and represent an area of the order of several centimeters on each side.

The image of FIG. 4B was obtained by placing a blue filter 22 over light source 20 to produce an incident beam of blue light. As described above, the relative intensity gains for the different color chips in the detector 30 were adjusted to maximize the detection of the returning fluorescent light and to reduce the detection of any background reflected light. Specifically, the gain of the green chip was adjusted relative to those of the blue and red chips to enhance the signal at approximately 530 nm.

The image FIG. 4B has a central diffuse bright fluorescence region: this area is highly dysplastic and has differentially accumulated more of the sodium fluorescein. The small bright irregular features in FIG. 4B are artifacts arising from reflection of some of the incident light from the surface of the colon, and amounting to the glistening of the surface. (Indeed, some glistening is also evident in the image of FIG. 4A.)

The image of FIG. 4C is of a portion of the tissue imaged in FIGS. 4A and 4B; the field of view is approximately 500 μm×500 μm so this image is enlarged relative to those of FIGS. 4A and 4B. The incident light was produced by laser 60 at 488 nm; return light was passed through a narrow band filter with a peak of approximately 530 nm before impinging on detector 68, so that only fluorescence emitted by the sodium fluorescein would be collected.

The focal plane of endoscope 10 is variable from effectively zero (i.e. to image the surface layer of the tissue) to a depth of about 250 μm below the surface of the tissue. In this example a focal plane approximately 50 μm below the surface of the tissue was employed. Consequently, this image contains shows structure and demonstrates the degree of cell dysplasia. The reflectance visible in FIG. 4B, since it is a surface effect, was not collected.

Since modifications within the spirit and scope of the invention may readily be effected by persons skilled within the art, it is to be understood that this invention is not limited to the particular embodiments described by way of example hereinabove.

In the claims that follow and in the preceding description of certain embodiments, except where the context requires otherwise due to express language or necessary implication, the word “comprise”, or variations such as “comprises” or “comprising”, is used in an inclusive sense, i.e. to specify the presence of the stated features but not to preclude the presence or addition of further features in various embodiments.

Further, any reference herein to prior art is not intended to imply that such prior art forms or formed a part of the common general knowledge.

What is claimed is:

1. An endoscope comprising:
a first light source configured to illuminate biological tissue with light;
a first detector configured to detect macroscopic images and fluorescent images from the tissue by reflected light and fluorescent light induced in said tissue;
a second light source configured to illuminate the tissue with light;
a confocal microscopic waveguide configured to supply light from said second light source to said tissue and to supply microscopic fluorescent images of said tissue; and
a second detector configured to detect the microscopic fluorescent images from said confocal microscopic waveguide;
wherein light from said first light source and light from said second light source are transmitted to said tissue along different optical paths.

2. The endoscope as claimed in claim 1, wherein said first light source comprises a white light source and a filter for changing effective illumination of said tissue through the visible spectrum, such that different tissue structures being illuminated by the changing wavelengths of the illumination light reflect varying amounts of light to said first detector.

3. The endoscope as claimed in claim 2, wherein said detector comprises a monochrome detector.

4. The endoscope as claimed in claim 1, wherein said first light source comprises a continuous white light source and said first detector is a color detector configured to detect various amounts of the different wavelengths of reflected light.

5. The endoscope as claimed in claim 1, further comprising a waveguide for supplying light from said first light source to said tissue.

6. The endoscope as claimed in claim 5, wherein said further waveguide comprises a single fiber or a fiber bundle for conducting light from said first light source to said tissue and the first detector is at a free end of the endoscope.

7. The endoscope as claimed in claim 5, wherein said further waveguide comprises a first waveguide configured to conduct light to a free end of the endoscope and a second waveguide configured to receive reflected light and convey the reflected light to said first detector.

8. The endoscope as claimed in claim 7, further comprising a filter configured to filter light received by said second waveguide, located either at said free end of said endoscope or between said first detector and said second waveguide.

9. An endoscope as claimed in claim 1, wherein said endoscope has a variable focal plane.

10. The endoscope as claimed in claim 7, wherein said first and second waveguides comprise single fibers or fiber bundles.

11. The endoscope as claimed in claim 1, wherein said confocal microscopic waveguide is a single fiber or fiber bundle.

12. The endoscope as claimed in claim 11, wherein said confocal microscopic waveguide includes a fiber coupler configured to receive fluorescent light and direct said fluorescent light to said second detector.

13. The endoscope as claimed in claim 1, including a monitor configured to provide a display that comprises an overlap of the macroscopic and fluorescent images to produce a single image.

14. The endoscope as claimed in claim 1, wherein said second light source produces light at a predetermined monochrome wavelength for passage through said confocal microscopic waveguide.

15. The endoscope as claimed in claim 1, wherein said second light source comprises a laser light source.

16. The endoscope as claimed in claim 1, wherein said first detector is connectable to a processor and said first detector includes a plurality of different color chips so that the intensity gains for said different color chips in said first detector are adjustable to maximize detection of returning fluorescent light and reduce detection of any background reflected light.

17. The endoscope as claimed in claim 16, wherein said second detector is connectable to said processor so that said processor can process said images and display on a monitor the light image, the macroscopic fluorescent image and the microscopic fluorescent image.

18. The endoscope as claimed in claim 1, wherein said fluorescent light is induced in said tissue by administering an exogenous fluorescent contrast agent to a patient.

19. A method of inspecting a patient's tissue by use of an endoscope, comprising:
applying an exogenous contrast agent to the patient;
illuminating said tissue with said endoscope;
detecting with said endoscope a light image of said tissue;
detecting with said endoscope a macroscopic fluorescent image; and
detecting with said endoscope a microscopic confocal fluorescent image of said tissue.

20. The method as claimed in claim 19, wherein the method further comprises controlling filter parameters and gain of a color detector of said endoscope to be at the maximum excitation and emission peaks respectively for said contrast agent.

21. The method as claimed in claim 19, further comprising inspecting the macroscopic fluorescent image to identify regions of interest and further inspecting microscopic confocal fluorescent images of those regions.

22. The method as claimed in claim 19, wherein applying said exogenous contrast agent to the patient comprises applying acriflavine.

23. An endoscope comprising:
a selector mechanism configured to select between a plurality of modes of operation, the modes of operation comprising:
a white light color mode of operation in which a color image of a patient's tissue is obtained;
a macroscopic fluorescent mode in which a macroscopic fluorescent image of the tissue is obtained; and
a microscopic confocal fluorescent mode in which a microscopic fluorescent image of said tissue is obtained.

24. The endoscope as claimed in claim 23, wherein said endoscope has a variable focal plane.

25. The endoscope as claimed in claim 23, wherein the selector mechanism comprises a filter configured to produce light of two or more selectable wavelengths based on the selected mode of operation.

26. The endoscope as claimed in claim 23, wherein the selector mechanism comprises a charge coupled device (CCD) configured to detect light of two or more selectable wavelengths based on the selected mode of operation.

27. A method of obtaining images of a patient with a single endoscope, the method comprising:
administering an exogenous contrast agent to the patient; and
obtaining one or more of a white light color image, a macroscopic fluorescent image and a microscopic confocal fluorescent image of tissue of said patient.
